(19) United States
(12) Patent Application Publication
(10) Pub. No.: US 2003/0170151 A1
(43) Pub. Date: Sep. 11, 2003

(54) Biohazard treatment systems

(76) Inventors: Charles Eric Hunter, Jefferson, NC (US); John H. Hebrank, Durham, NC (US); Laurie E. McNeil, Chapel Hill, NC (US); Drew G. Narayan, Durham, NC (US); Michael A. Wiener, New York, NY (US)

Correspondence Address: Finnegan, Henderson, Farabow,, Garrett & Dunner, L.L.P., 1300 I Street, N.W., Washington, DC, 20005-3315, US

(21) Appl. No.: 10/259,463

(22) Filed: Sep. 30, 2002

Related U.S. Application Data
(60) Provisional application No. 60/362,393, filed on Mar. 8, 2002.

(51) Int. Cl. B01J 19/08

(52) U.S. Cl. 422/186.3; 588/204

(57) ABSTRACT

Systems and methods for exposing fluids and other materials that may contain biohazards to ultraviolet radiation are provided. One system for exposing a fluid includes a baffled conduit for conveying the fluid so that the fluid flow and its exposure to ultraviolet radiation is rendered more uniform. Other systems include feedback for determining when to replace light sources and filters and to ensure proper biodosimetry. Additional methods and systems for exposing fluids and other materials that may contain biohazards are also provided.

### CROSS REFERENCE TO RELATED APPLICATION

This claims priority to U.S. Provisional Patent Application No. 60/362,393, filed Mar. 8, 2002, which is hereby incorporated by reference herein in its entirety.

### FIELD OF THE INVENTION

This invention relates to systems and methods for exposing materials to ultraviolet radiation and more particularly to neutralizing biohazards located on or within the material, which may be a solid, liquid, or gas.

### BACKGROUND OF THE INVENTION

Certain biological microorganisms can have significant negative affects on the health of humans and animals. Such organisms include, for example, common mold spores and pollen, as well as more deadly biological hazards, such as anthrax and small pox. As used herein, the term “biohazard” refers to all types of biological microorganisms that have negative side affects, including potentially deadly microorganisms.

The use of ultraviolet germicidal irradiation for the killing of microorganisms and biohazards is known (“Ultraviolet Germicidal Irradiation,” http://www.engr.psu.edu/ae/wjk/wjkuvgi.html, printed and downloaded on Sep. 11, 2002). As used herein, the terms “inactivated,” “deactivated,” “killed,” and “neutralized” all describe the condition when a biohazard loses its ability to reproduce. Inactivation is known to be a stochastic process and is generally measured statistically.

Conventional methods and systems are often ineffective at inactivating these hazards because they are unable to expose the materials that contain these biohazards to a minimum radiation dose.

Moreover, although microbes are vulnerable to the effects of ultraviolet light at wavelengths at or near 253.7 nm (due to the resonance of this wavelength with various molecular structures, including proteins) within the biohazards, vulnerability can depend on wavelength. For example, it is known that the bactericidal action of ultraviolet radiation of different wavelengths in Staphylococcus aureus cells closely match the absorption spectra of its nucleotide bases (Diffey, B. L., “Solar ultraviolet radiation effects on biological systems,” Review in Physics in Medicine and Biology Vol. 36 No. 3, at 299-328 (1991)). Conventional deactivation methods normally use, however, a single ultraviolet radiation source (e.g., a mercury-vapor lamp) to inactivate many types of cells, viruses, and bacteria, irrespective of the particular species being targeted.

Also, conventional techniques for treating surfaces are often ineffective because the apparatus are insufficiently mobile to direct the radiation as necessary. Furthermore, conventional methods and systems for treating fluids, such as air, with ultraviolet radiation are often ineffective because the biohazards are distributed non-uniformly within the fluid being treated.

### SUMMARY OF THE INVENTION

Consistent with the invention systems and methods are provided for substantially neutralizing biohazards in a variety of materials. These methods and systems expose materials to radiation from one or more light-emitting devices that emit short-wavelength radiation for reducing, neutralizing, and substantially inactivating, biohazards in those materials.

In one embodiment, a system is provided for exposing a fluid, that may contain a biohazard, to ultraviolet radiation. The system can include a conduit for conveying the fluid, wherein the conduit has an input, an output, a length, and a cross section along its length. The fluid has a distribution in the conduit, which is baffled so that the fluid flow is rendered more uniform while being conveyed through the conduit. The system can also include at least one array of solid-state light-emitting devices mounted to emit short-wavelength radiation in the conduit for neutralizing the biohazard.

In another embodiment, a system includes a conveyor for conveying the material, at least one array of solid-state light-emitting devices mounted to emit short-wavelength radiation at the material while being conveyed by the conveyor, wherein the radiation has an intensity along the length of the conveyor, at least one photodetector positioned to monitor the intensity of the devices, and a conveyor controller for adjusting the speed of the conveyor such that the material is exposed to a predetermined radiation dose sufficient to neutralize the at least one biohazard, wherein the adjusting is based on an output of the at least one photodetector.

In yet another embodiment, a mobile system for exposing a material to a directed beam of ultraviolet radiation is provided. The mobile system includes at least one mobile array of solid-state light-emitting devices mounted to emit short-wavelength radiation in the form of a beam having a direction. The mobile system also can include a controller for adjusting at least the direction of the beam such that the material is exposed to a predetermined radiation dose sufficient to neutralize the at least one biohazard.

In a further embodiment, a system is provided for exposing a surface to a directed beam of ultraviolet radiation. The system can include a light source for emitting short-wavelength radiation in a direction, a micro-mirror device having a plurality of independently controllable mirrors, each of the mirrors having a high reflectivity at the short-wavelengths, a waveguide having an input positioned to receive at least a portion of the radiation and an output positioned to direct the radiation toward the micro-mirror device, and a micro-mirror device controller coupled to the micro-mirror device for controlling the orientation of the mirrors such that the surface is exposed to a predetermined radiation dose sufficient to neutralize the at least one biohazard. It will be appreciated, however, that a macro-mirror device, which may contain one or more mirrors, can be used instead of the micro-mirror device.

In still another embodiment, an apparatus for attenuating ultraviolet-light emission for use with a system that inactivates biohazards using an ultraviolet light source is provided. The system has an ultraviolet light-absorbing surface disposed on an inner surface of the conduit or on a filter for use with such a system.

In yet another embodiment, a system is provided that includes a conduit that conveys air and at least one array of light-emitting devices mounted to emit short-wavelength radiation in the conduit for neutralizing the biohazard. The array includes at least two different types of ultraviolet light-emitting devices. A first type of device has a peak wavelength that is different from a second type of device.

In still another embodiment, a system is provided for exposing air to ultraviolet radiation in a killing zone of a conduit. The system has an array of light-emitting devices mounted to emit short-wavelength radiation in the conduit for neutralizing the biohazard and at least one photodetector located in the conduit to sense an ultraviolet radiation intensity and generate a signal indicative of the ultraviolet radiation. The system also includes a unit for determining, based on the at least one photodetector signal, whether any of the light-emitting devices require service.

In another embodiment, an ozone reactive surface is provided for use with an air processing system that inactivates airborne biohazards using an ultraviolet light source. The ozone reactive surface includes an unsaturated organic polymer, a metal sulfide, a metal hydroxide, or any combination thereof.

Methods for exposing various materials to substantially uniform and/or predetermined doses of ultraviolet radiation are also provided.

### BRIEF DESCRIPTION OF THE DRAWINGS

Advantages of the invention will be apparent upon consideration of the following detailed description, taken in conjunction with the accompanying drawings, in which like reference characters refer to like parts throughout, and in which:

FIG. 1 shows a simplified illustrative system for exposing biological hazards that may be present in fluids, such as liquids and gases, to short-wavelength (ultraviolet) radiation consistent with this invention;

FIG. 2 shows a cross-sectional view of a conduit that includes a variable cross-sectional portion consistent with invention;

FIG. 3 shows a simplified planar view of a centrifugal-force sorting device consistent with this invention;

FIG. 4 shows another illustrative system for exposing biological hazards that may be present in fluids to short-wavelength radiation consistent with this invention;

FIG. 5 shows a planar view of an illustrative two-dimensional array of ultraviolet LEDs that can be used as a light source consistent with this invention.

FIG. 6 shows an illustrative system for exposing biological hazards that may be present in air to short-wavelength radiation consistent with this invention;

FIG. 7 shows a conduit in an illustrative system for exposing biological hazards to short-wavelength radiation and an illustrative two stage removable filter consistent with this invention;

FIG. 8 shows another illustrative system for exposing biological hazards to short-wavelength radiation, including a conduit having a killing zone, an illustrative three stage removable filter, and an illustrative apparatus for cleaning the surfaces of the light sources located within the killing zone consistent with this invention;

FIG. 9 shows an illustrative conduit that attenuates ultraviolet light with a coating consistent with this invention;

FIG. 10 shows an illustrative attenuating screen to attenuate (e.g., filter) extraneous ultraviolet light rays from reaching port consistent with this invention;

FIG. 11 shows an illustrative system in which a killing zone includes at least one solid-state light-emitting diode and at least one mercury vapor lamp;

FIG. 12 shows illustrative normalized ultraviolet radiation spectra on an arbitrary wavelength scale that could be generated by different light-emitting devices within a killing zone consistent with this invention;

FIGS. 13 and 14 show composite ultraviolet spectra formed by different combinations of spectra shown in FIG. 12 consistent with this invention;

FIG. 15 shows yet another illustrative embodiment for exposing biological hazards that may be present in materials, such as solid objects, to short-wavelength radiation consistent with this invention;

FIG. 16 shows a mobile system for exposing a material to directed beam of ultraviolet radiation consistent with this invention;

FIG. 17 shows a hand-held device for exposing material to a directed beam of ultraviolet radiation consistent with this invention;

FIG. 18 shows still another illustrative system for exposing a potentially contaminated surface to a directed beam of ultraviolet radiation consistent with this invention;

FIG. 19 shows a perspective view of an illustrative device for exposing biological hazards that may be present on surfaces that is mounted on a mobile vehicle that moves along a track consistent with this invention;

FIG. 20 shows an illustrative cross section of the track and mobile vehicle shown in FIG. 19, including a waveguide and a roller driving means consistent with this invention; and

FIG. 21 shows a planar view of an illustrative system that includes diodes (or clusters of diodes) for emitting ultraviolet radiation and a strip onto which the diodes are mounted consistent with this invention.

### DESCRIPTION OF THE EMBODIMENTS

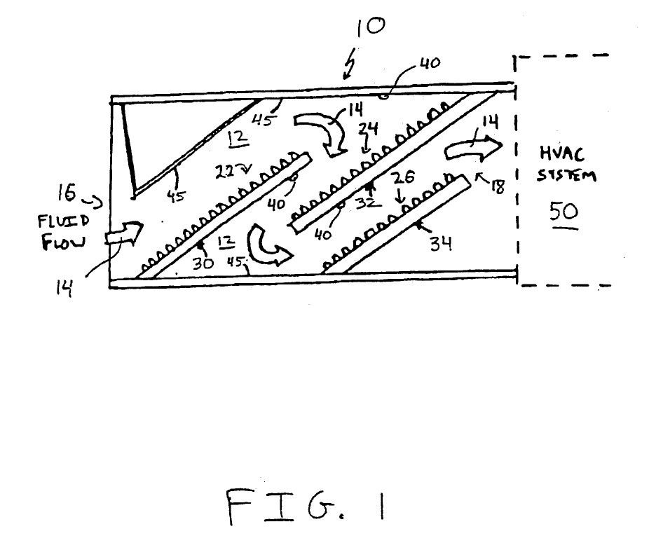

FIG. 1 shows an illustrative system 10 consistent with this invention for exposing biological hazards that may be present in fluids, such as liquids and gases, to short-wavelength (ultraviolet) radiation. As used herein, biohazards can include mold spores, microorganisms, and other biological organisms that are potentially harmful to humans and other animals. Short-wavelength radiation includes radiation having a wavelength that is less than about 410 nm.

System 10 includes a conduit 12 for conveying a fluid 14 and at least one array 22, 24, and 26 of solid state light-emitting devices. Conduit 12 has an input 16 coupled to a source of fluid (not shown) and an output 18. Conduit 12 is baffled so that fluid 14 flows more uniformly while being conveyed through conduit 12. The light-emitting devices of arrays 22, 24, and 26 are mounted in the conduit such that they emit sufficient radiation in conduit 12 for neutralizing biohazards, thus forming a “killing zone” in the conduit. A suitable electronic controller and power supply may also be included. In an embodiment, the power supply can supply the approximately 10 Volts typically required by the solid-state light-emitting devices. Additionally, the controller can include a cycle timer that periodically, or at predetermined times, controls a power MOSFET, or similar power switching device, to supply current to the light-emitting devices.

In one embodiment, system 10 can include one or more baffles 30, 32, and 34 onto or into which arrays 22, 24, and 26 can be mounted. Alternatively, conduit 12 itself can be made to follow a circuitous route, thereby eliminating the use of baffle elements 30, 32, and 34, yet still obtaining the benefit of causing fluid 14 to flow more uniformly, allowing for more uniform irradiation thereof. Two or more arrays can be arranged such that they are not coplanar, requiring the fluid path to be more circuitous, as shown in FIG. 1.

System 10 can be coupled in series to any type of a fluid processing apparatus 50 for causing the fluid to flow through conduit 12 and receive an appropriate dose of radiation. The fluid processing apparatus can be, for example, a gas or liquid (e.g., air or water) heating apparatus, ventilating apparatus, conditioning apparatus, filtering apparatus, cleaning apparatus, and any combination thereof. The system can be coupled in series either before or after the fluid processing apparatus, as the particular application requires.

To improve the effectiveness and efficiency of system 10, an inner surface 45 of conduit 12 can be made highly reflective for the short wavelengths used to neutralize the biohazards. High reflectivity can be achieved using conventional UV coating techniques or by using materials, such as metals, that are known to have a relatively high UV reflectivity. In one embodiment, surface 45 has at least a 50% reflectivity for UV radiation.

The electrical power supplied to the LED arrays can be used as an indication of the optical power emitted by them, thus providing a technique for estimating and monitoring the actual UV radiation dose incident on the fluid passing through conduit 12. As explained more fully below, however, dust and other airborne matter can deposit on the surface of the arrays, decreasing their effectiveness. The rate at which fluid 16 flows through conduit 12 can also be varied by varying the position and orientation of baffles 30, 32, and 34. Fast rates tend to decrease the dose while slower rates tend to increase the dose. Thus, the electrical power level can be used in a feedback loop to control the dose by varying the position or orientation of one or more of baffles 30, 32, and 34.

As an alternative to using the electrical power level supplied to the light-emitting devices, one can use a measured radiation intensity by placing one or more photodetectors 40 in conduit 12. Photodetectors 40 generate signals indicative of the measured radiation intensities at different locations along conduit 12 and can thus be used as a way to accurately determine the actual radiation dose. Thus, these signals can also be used in a feedback loop to control the fluid flow through conduit 12. Photodetectors consistent with this invention can use SiC, although other materials can also be used.

System 10 can further include a power supply (not shown) that can supply power at a sufficient voltage to drive the at least one array of devices and a controller (not shown) for controlling the power to the devices. The controller can include a cycle timer that controls a power switching device used to supply power to the devices. The timer can supply power to the devices periodically, at predetermined times, or it can supply power to the devices according to both techniques.

As briefly explained above, the fluid flow can be controlled in the conduit such that the fluid is exposed to a predetermined radiation dose, that is, exposed to a predetermined radiation intensity for a predetermined period of time. Thus, system 10 can include at least one photodetector positioned to monitor the intensity and a flow controller for adjusting the speed of the fluid in the conduit such that the fluid is exposed to a sufficient radiation dose to neutralize the at least one biohazard. In this case, the flow adjustment can be based at least on an output of the at least one photodetector. In addition to the flow, photodetector signals can be used increase the power supplied to the light emitting devices as the devices become less efficient with age to ensure proper UV treatment. The photodetector signals can also be used to trigger alarms if the light emitting devices burn out or require too much power to sustain a particular intensity level.

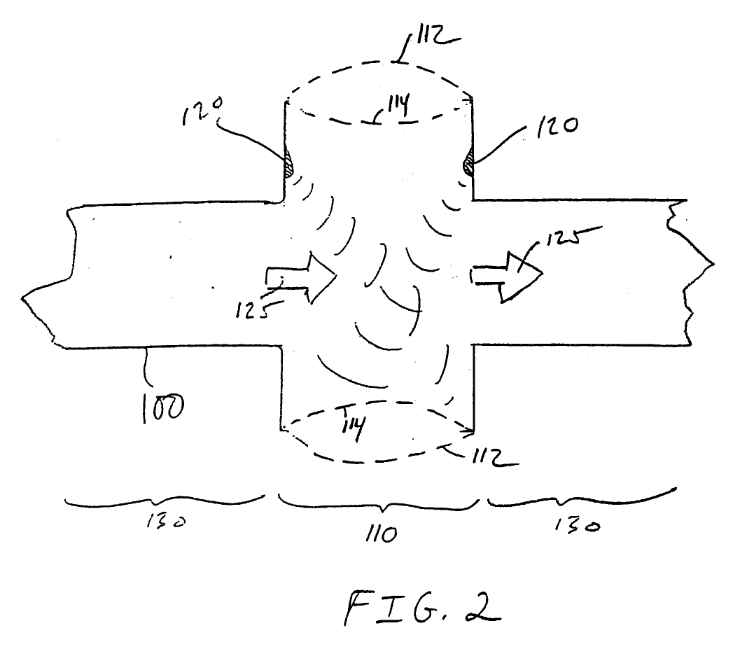

In one embodiment, the cross section of a fluid conduit can be variable. Cross-section variability enables one to vary the dose delivered to the fluid in the killing zone of the system. FIG. 2, for example, shows a cross-sectional view of conduit 100, which includes a variable cross sectional portion 110. Portion 110 can be formed from a flexible material that can increase (or decrease) its effective diameter from a position 114 to a position 112, for example. It will be appreciated, however, that any convenient technique for varying the diameter of the killing zone (i.e., portion 110) can be used consistent with this invention. Portion 110 can have one or more UV light-emitting devices 120 mounted in or near the portion. The fluid, indicated by arrows 125, will slow in portion 110 if the cross section of portion 110 is greater than the cross-section of adjacent regions 130. Alternatively, the fluid flow will increase in portion 110 if the cross section of that portion is less than the cross section of adjacent regions 130.

The cross-section of portion 110 can be controlled by a cross-sectional controller (not shown) for adjusting the cross section of portion 110 such that the fluid is exposed to a sufficient radiation dose to sufficiently neutralize any biohazards that may be present in the fluid. The controller and/or the voltage regulator can be based, for example, on signals provided by photodetectors or the electrical power levels supplied to the light-emitting devices.

In another embodiment consistent with this invention, a system for exposing fluids (or powders) to ultraviolet radiation can include a sorting device for physically segregating the fluid into at least a first constituent part and a second constituent part. Then, one or more arrays of light-emitting devices can be mounted such that the first part is exposed to a higher radiation intensity than the second part.

FIG. 3, for example, shows a simplified planar view of a centrifugal-force sorting device 130 consistent with this invention. Device 130 includes an input 132, an output 134, and a centrifugal chamber 136. During operation, chamber 136 causes a fluid, represented by single head arrows 140, to rotate within chamber 136, thereby causing the fluid to separate into at least a first less dense constituent part and a second more dense constituent part along a radial gradient 142. Light-emitting devices 144 can be congregated at any radial position to expose a particular part that has a particular density to more radiation than another part. Alternatively, light emitting devices 144 can be distributed evenly in a radial fashion and then used selectively to target different density constituent parts. It will be appreciated that similar devices, based on electric-fields, electromagnetic fields, magnetic fields, gravitational fields, porous screens, and any combination thereof, can also be used that sort fluids and powders according to this invention.

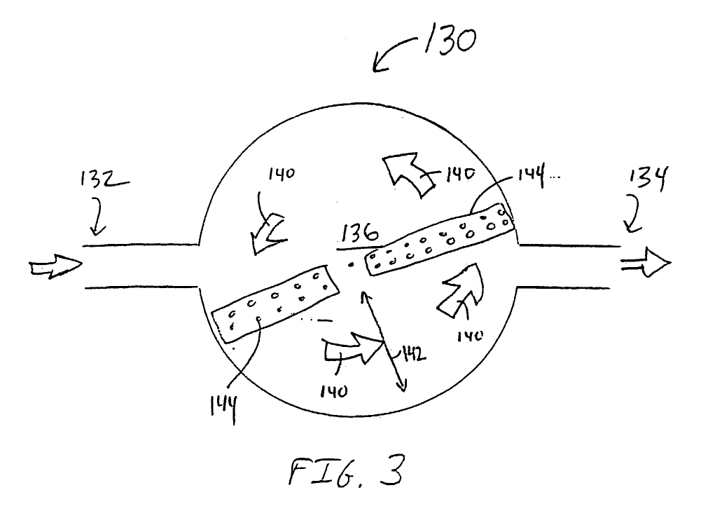

FIG. 4 shows another illustrative system 200 for exposing biological hazards that may be present in fluids to short-wavelength (ultraviolet) radiation. System 200 can include a conduit 202 and a conduit 204 for conveying a fluid and arrays 220, 222, 223, and 224 of solid state light-emitting devices. Conduits 202 and 204 share an input 216 and an output 218. Conduit 202 can be baffled so that the fluid flows more uniformly while being conveyed through conduits 202 and 204. The light-emitting devices of arrays 220, 222, 223, and 224 are mounted in the conduits such that they emit enough radiation to sufficiently neutralize any biohazards that may be present in the fluid. Once again, feedback may be used (e.g., with photodetectors and baffles or conduits with variable cross sections) to control the fluid flow through or radiation intensity within conduits 202 and 204, if desired, to obtain a particular dose.

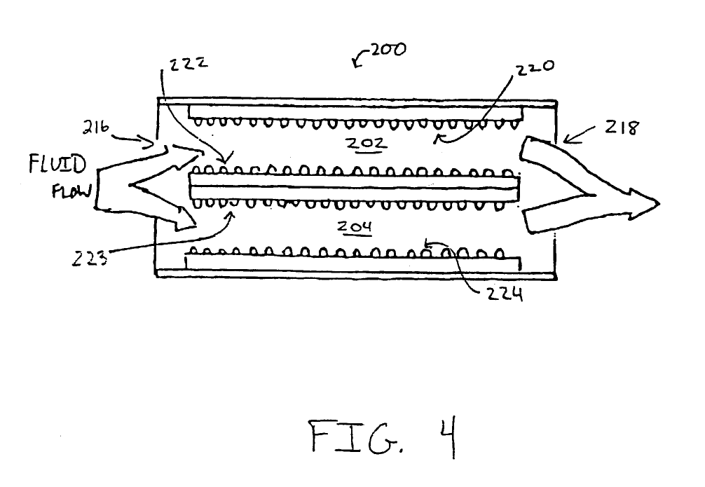

FIG. 5 shows illustrative two-dimensional array 500 of ultraviolet LEDs that can be used as a light source consistent with this invention. It will be appreciated that array 500 need not be planar, but could be in any convenient shape, including a shape that conforms to the inner surface of a system conduit. Consistent with this invention, the light-emitting devices 502 of array 500 can be mounted to emit short-wavelength radiation in the conduit for neutralizing one or more biohazards. When more than one type of biohazard is targeted, array 500 comprises at least two different types of ultraviolet light-emitting devices having different peak wavelengths.

Array 500 includes N types of devices, each having different peak wavelengths. For example, as shown in FIG. 5, array 500 includes a first type of device that has a first peak wavelength λ₁, a second type of device can has a second peak wavelength λ₂, and so on. Array 500 includes ten columns of nine types of devices. It will be appreciated, however, that array 500 can include any number of each type of device and that they need not be oriented in columns or rows.

A system for exposing air to ultraviolet radiation can include, for example, array 500. As shown in FIG. 6, the system can include a conduit 505 for conveying air from a first point 507 along its length to a second point 508. The system shown in FIG. 6 can include a power controller 512 for supplying power to each of light-emitting devices 502 according to a power distribution profile.

The power distribution profile defines the power supplied to each of the light-emitting devices of the array or to any other type of light source used by the system. The profile may be time-dependent (e.g., when pulsed light is desirable) and/or wavelength dependent (e.g., when different biohazards are believed to be present at different times). The power distribution profile can be selected, for example, from a look-up table stored in a memory unit. For example, when the system includes a biohazard detector 510, that detector can generate a signal that is transmitted to power controller 512. Based on that signal, power controller 512 can select a predetermined profile that matches the spectral sensitivity of the targeted biohazard (see, Cabaj et al., “The spectral UV sensitivity of microorganisms used in biodosimetry,” Water Science and Technology: Water Supply Vol. 2, No. 3, at 175-181 (2002)). The power distribution profile can also include dose information because different types of microorganisms often require different UV doses to be deactivated (see, “Some Micro-Organisms Deactivated By Ultraviolet Germicidal Light,” http://ultraviolet.com/microorgan.htm, printed and downloaded on Sep. 10, 2002). By adjusting the power distribution profile periodically or continually during operation, efficient and effective biohazard deactivation can be accomplished.

Biohazard detectors that can be used consistent with this invention can operate, for example, on fluorescent emission signature principles. Anthrax spores are known, for example, to fluoresce when exposed to certain ultraviolet light wavelengths (see “Team to build compact warming system for anthrax, other bioagents,”http://www.brown.edu/Administration/News Bureau/2001-02/01-156.html, downloaded and printed on Sep. 9, 2002). In fact, many biohazards can be identified by their spectroscopic fingerprints. By detecting this fluorescence with at least one photodetector, which may be a spectroscopic device, it is possible to generate a signal identifying a particular biohazard that can be sent to power controller 512 for selecting a predetermined power distribution profile.

In one embodiment, a biohazard detector can be coupled to the power controller through a communication network, such as the Internet. In this way, sophisticated detectors that may be too expensive for use in most individual homes can be shared. Then, a single detector could be programmed to send biohazard detection signals to multiple residential homes and industrial facilities. These detection signals would then cause distributed power controllers 512 to either select or generate an appropriate power distribution profile. It will further be appreciated that power controller 512 can be manually operated, if desirable, though a manual interface 520.

The power distribution profile can also be determined in real-time based on one or more inputs. For example, the system can include an ambient condition monitor 515 that can monitor one or more environmental ambient conditions, such as humidity and temperature. The measured condition, then, can be used by the power controller to calculate a precise power distribution profile that would be optimized for that condition. For example, higher humidity levels may correspond to higher airborne mold concentrations. In this case, the power distribution profile may cause power controller 512 to supply a relatively high power-level to appropriate light-emitting devices. An appropriate light-emitting device may be one that has a peak wavelength that corresponds to a maximum sensitivity for mold. It will also be appreciated that any predetermined power distribution profile can be modified by ambient condition information.

It will be further appreciated that the temperature of the light-emitting devices can also be monitored with one or more temperature sensors. The temperature information provided by the sensors can be used to adjust the power supplied to each of the devices, which may be highly temperature dependent. Thus, the temperature information can be used to select or determine, empirically or analytically, an appropriate power distribution profile.

FIG. 12 shows normalized ultraviolet radiation spectra that could be generated by different light-emitting devices within a killing zone consistent with this invention. The spectra are expanded vertically for illustrative clarity. Spectrum 730 is a relatively narrow, high energy spectrum that could be generated, for example, by a mercury-vapor lamp (e.g., spectrum 730 can correspond to the 253.7 nm line). Spectra 735, 740, 745, 750, and 755 are wider than spectrum 730 and could correspond to the spectral outputs light-emitting diodes made, for example, with AlGaN, AIN, or any other suitable material (see, e.g., TABLE I). It will be appreciated that any number of light-emitting devices can be used consistent with this invention and that two or more devices in a single killing zone can have the same or substantially the same spectrum. Such spectral redundancy can be useful when the period between maintenance calls is greater than the anticipated lifetime of any individual device.

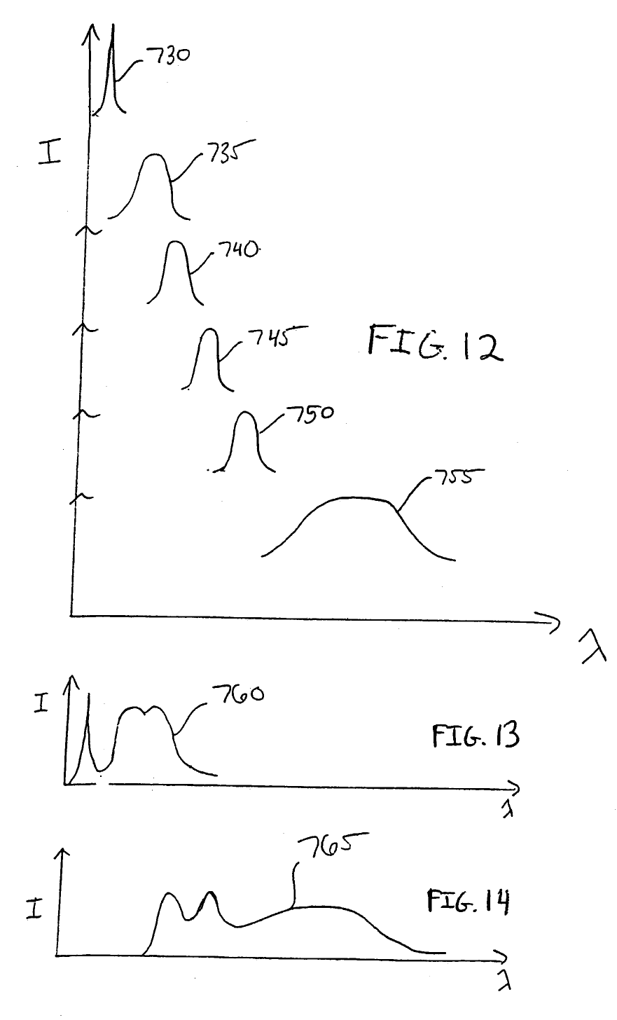

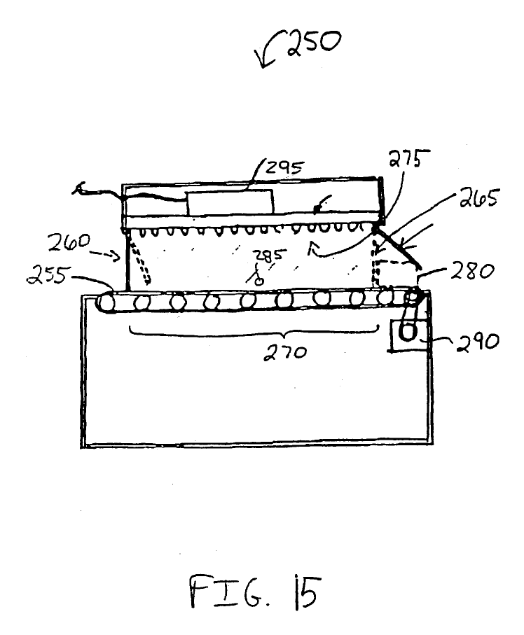

FIG. 13 shows composite ultraviolet spectra 760 formed by combining spectra 730, 735, and 740. Similarly, FIG. 14 shows composite ultraviolet spectra 765 formed by combining spectra 745, 750, and 755. It will be appreciated that the relative intensity of each component of spectra 760 and 765 can be determined by a particular power distribution profile.

As mentioned above, array 500 can include at least two different types of light-emitting devices with different peak wavelengths. In one embodiment, a first type of device has a first peak wavelength in a first range between about 260 nm and about 280 nm and a second type having a second peak wavelength in a second range between about 280 nm and about 300 nm. In another embodiment, both types of devices have peak wavelengths in a range between about 260 nm and about 280 nm. Generally, the system has a wavelength treatment range between a lower limit and an upper limit. Then, each type of device can have a different peak wavelength that is distributed between the lower and upper limits. AlGaN and AIN-based light-emitting diodes are believed to be particularly well suited for both of these wavelength ranges, although other types of light sources can be used.

It will be appreciated that different types of devices having different peak wavelengths can be operated simultaneously or sequentially. In either case, the effective spectral distribution can be adjusted by supplying different power levels to different devices.

The system shown in FIG. 6 can also include at least one photodetector 525 located in or adjacent to conduit 505 to sense the ultraviolet radiation intensity and generate a signal indicative of the ultraviolet radiation flux. The system can further include a unit 530 for determining, based on the photodetector signal(s), whether any of light-emitting devices 502 require service. It is determined that service is required, a maintenance signal can be transmitted by transmitter 535 to a maintenance service 540. The maintenance signal can include information indicative of the particular service that must be performed.

In one embodiment, the system can include a filter 550 that is located in series with the killing zone of the conduit. Filter 550 can be placed upstream or downstream of the killing zone, but is preferably upstream to prevent dust and other particles from attaching to array 500 of light-emitting devices 502. Once attached, these particles can reduce the effectiveness of the killing zone by blocking the ultraviolet light.

In addition to the filter, the system can include a unit 530 for determining whether the filter requires replacement. As shown in FIG. 6, the unit can be the same as the unit used to determine whether any of light-emitting devices 502 require service. Thus, the unit can be coupled to transmitter 535 for transmitting a replacement signal to replacement service 540 if the filter were determined to require replacement. Filter 550 can provide a status signal to unit 530 using wireless or wired coupling. Alternatively, it will be appreciated that unit 530 can be on board filter 550 and therefore replaced when filter 550 is replaced.

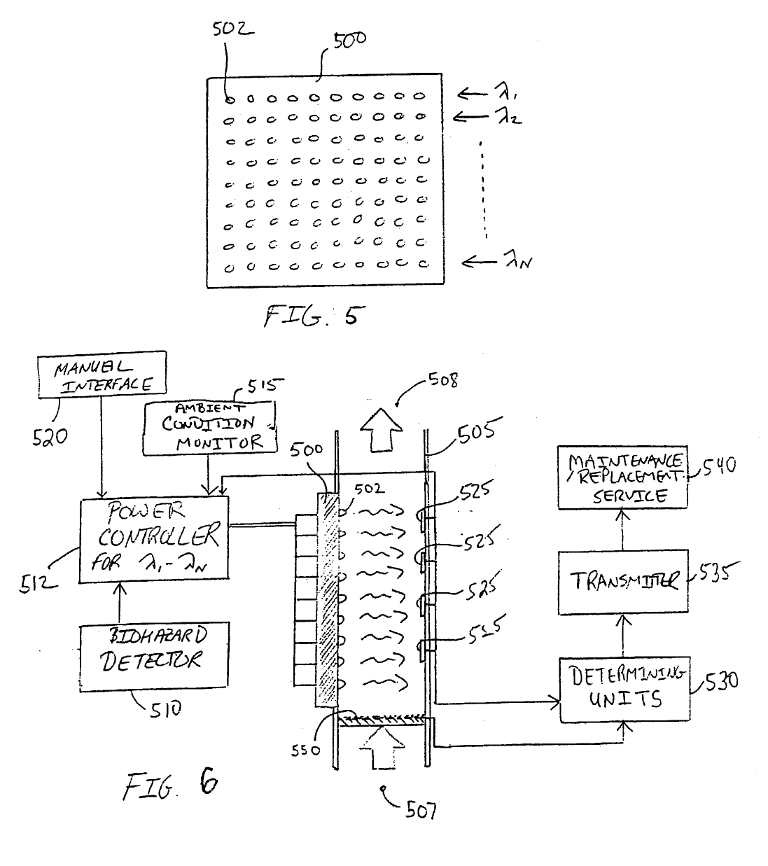

Unit 530 can determine whether filter 550 must be replaced in a number of ways. A first way involves the use of a dust monitor on the filter. FIG. 7, for example, shows conduit 600 and removable filter 610. Filter 610 can include one or more stages, but preferably includes at least two stages 612 and 614. First stage 612, for example, can include a light-emitting diode 615, light-detecting photodetector 620, and reflective surface 625. During operation, diode 615 directs a beam 630 of light toward surface 625. Surface 625 then reflects beam 630 toward photodiode 620. Filter 610 can also include power supply 635 for powering diode 615 and photodetector 620, including any additional circuitry (not shown) that may be desirable to amplify and analyze the signal generated by photodetector 620. Over time, reflective surface 625 will become coated with dust and other particles, degrading the intensity of reflected beam 631. Thus, photodetector 620 of the dust monitor can generate a status signal indicative of an amount of dust trapped by the filter. Then, the status signal can be provided to a transmitter (e.g., transmitter 535) when the amount of dust trapped by filter 610 exceeds a predetermined threshold amount.

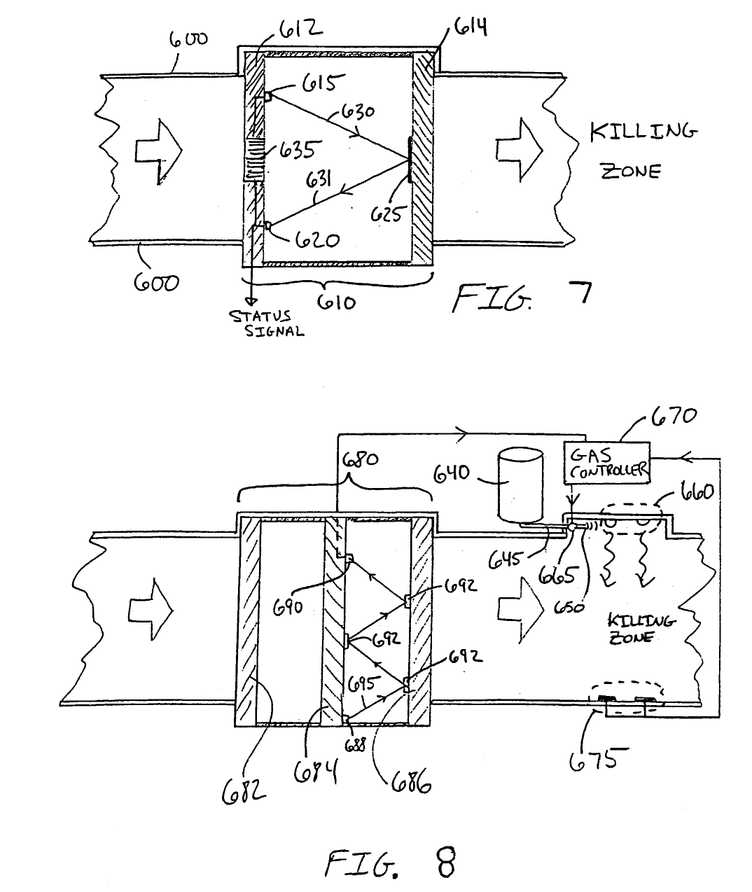

If it is determined that the array of light-emitting devices requires service, the system can include a self-cleaning apparatus. As shown in FIG. 8, the self-cleaning apparatus can include a tank 640 that includes a fluid under pressure, a pipeline 645 in fluid communication with the fluid in tank 640, a fluid spout 650 connected to pipeline 645 that can direct the fluid toward the array 660 of light-emitting devices, a fluid valve 665 for limiting fluid flow in pipeline 645, and a fluid controller 670, which may be coupled to at least one photodetector 675 and fluid valve 665. Photodetector 675 can generate and send a status signal to fluid controller 670 which opens and closes fluid valve 665. The fluid used to clean the light-emitting devices can be, for example, a gas, such as nitrogen or dry air, or an organic solvent.

FIG. 8 also shows a three stage filter 680 that includes stages 682, 684, and 686. Like filter 610, filter 680 can include a light-emitting diode 688 and a light-detecting photodetector 690. But, rather than including a single reflective surface, filter 680 includes multiple reflective surfaces 692. During operation, diode 688 directs a beam 695 of light toward surfaces 692. Surfaces 692 reflect beam 695 toward photodiode 690. Filter 680 can also include a power supply for powering the light-emitting diode, the photodetector, and any additional circuitry (not shown) that may be desirable to amplify and analyze the signal generated by the photodetector. Over time, reflective surfaces 692 will become coated with dust and other particles, degrading the intensity of beam 695 upon reflection by those surfaces. Thus, photodetector 690 can generate a status signal indicative of an amount of dust trapped by the filter, which corresponds to the amount of beam degradation).

Another way that unit 530 can determine whether filter 550 must be replaced involves monitoring an intensity of transmitted light (e.g., the ultraviolet light in the killing zone) using photodetectors 525. Rather than monitoring a reflected signal within or on the surface of filter 610, this technique can involve monitoring the intensity of the transmitted light upstream and downstream from the filter and comparing the transmission measurements. As the filter becomes less effective, the difference between the downstream and upstream transmission measurements would change. Then, when the difference is sufficiently different, it could trigger unit 530 to generate a maintenance or replacement signal to transmitter 535.

Rather than comparing two or more transmission measurements, one or more light transmission levels can be measured across the conduit downstream of the filter. Using this technique, the levels (or an average thereof) are compared to a predetermined threshold, rather than another upstream measurement, to determine whether the filter requires replacement. To ensure accuracy, the threshold can be determined using a calibration step in which a clean gas is passed through the conduit.

Yet another technique for determining whether filter 550 must be replaced involves integrating the optical intensity difference between the upstream and downstream transmission measurements. The larger the difference at any given time, the more particulate matter that is being trapped by the filter. When the difference is integrated over time, it represents a total amount of matter trapped by the filter. When that amount is greater than a predetermined threshold amount, unit 530 can, in one embodiment, cause transmitter 535 to notify filter replacement agent 540 to replace the filter or simply notify, for example, a home owner or maintenance person.

In addition to removing airborne matter, the filter can perform a number of additional roles, including the removal of ozone. For example, it is known that mercury-vapor lamps have been used to generate high-energy ultraviolet light, but such lamps can have the undesirable side-effect of generating ozone. Although not wishing to be bound by any particular theory, it is believed that high energy atomic transitions in vaporized mercury atoms, which emit radiation having wavelengths below 242 nm, can cause harmless oxygen molecules (i.e., O₂) to dissociate and become harmful ozone (i.e., O₃) (see, Diffey). It is known, however, that ozone attacks or “oxidizes” human lung tissue and therefore should be avoided. Thus, the use of ultraviolet light having wavelengths below about 242 nm to kill airborne biohazards could generate ozone and pose a health risk.

Thus, a system consistent with this invention can include a filter, or an inner surface of an air conduit, that has an ozone reactive surface that converts ozone into a less harmful molecule. Many substances are known to react strongly with ozone. For example, most unsaturated organic compounds will be attacked by ozone, thereby reducing the ozone level. Water and ozone are known to also combine readily, which is why ozone is often used to clean contaminated water. Metal sulfides and hydroxides also react strongly with ozone: PbS+4 O₃→PbSO₄+4 O₂. Also, KOH can be used in a catalytic reaction as follows:

2 KOH+5 O₃→2 KO₃+5 O₂+H₂O

KO₃+H₂O→KOH+O₂+{OH}

2 {OH}→H₂O+0.5 O₂

The above reaction also works with other metal hydroxides.

Thus, consistent with this invention, a filter or conduit surface can be coated with an unsaturated polymer, such as polyisoprene, although such polymer may get brittle over extended ultraviolet light exposure. Such coatings could be deposited from solution.

Alternatively, PbS can be sprayed on in powder form. Eventually, the coating change into PbSO₄, but it would still be a solid and would remain on the ductwork. In another embodiment, the ozone reactive coating could include a metal hydroxide, which could be applied using a pickling process (i.e., in a chemical bath). It is believed that a metal hydroxide would be particularly robust over time. It will be appreciated, then, that an ozone filtering system consistent with this invention can remove ozone that may be present in the air before treatment, as well ozone created by the ultraviolet light treatment itself.

As described above, a system that uses ultraviolet light to treat biohazards can be coupled in series with any type of fluid processing apparatus, such as, for example, a heating, ventilating, and air conditioning (“HVAC”) apparatus. The ultraviolet light, however, can be internally reflected by the conduit and emerge at one or more inputs or outputs (e.g., vents) of the system. If the system is for use in a residential system, for example, the emerging ultraviolet light create a safety hazard to anyone at or near the inputs or outputs of the system.

Thus, an apparatus is provided for attenuating ultraviolet-light emission from a system that inactivates biohazards using ultraviolet light. The apparatus includes an ultraviolet light-absorbing surface disposed on an inner surface of the conduit or on a replaceable filter. The ultraviolet light-absorbing surface can be a roughened surface that substantially diffuses the light. The roughened surface can be formed by chemically etching the inner surface of the conduit or coating the inner surface with an ultraviolet light-absorbing material.

In one embodiment, the coating can include a powder and a binding material. The binding material can be an adhesive, a resin, or any other carrier that is capable of holding the powder in place. The coating mixture can, for example, be sprayed or brushed on to the inner surface of the conduit. It will be appreciated, however, that the powder can be bound to an intermediate material, such as a film or paper, which can then be attached to the inner surface of the conduit. FIG. 9 shows one embodiment of conduit 230, in which reflected ultraviolet light rays 232 are attenuated upon reflection by coating 234 to become attenuated light rays 236 and 238.

In one embodiment, the length scale of the powder is on the order of the wavelength of the ultraviolet light being attenuated. The powder can be any material that is substantially stable upon extended exposure to the ultraviolet light, such as inorganic materials. Some of the inorganic materials that can be used consistent with this invention are silicate glass powders, ceramic powders, or combinations thereof.

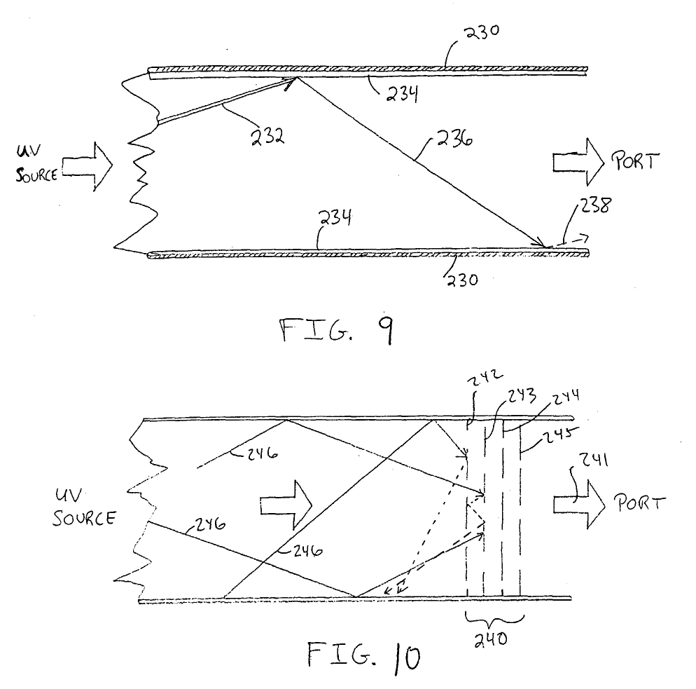

FIG. 10 shows another embodiment consistent with this invention in which screen 240 can be used to attenuate (e.g., filter) extraneous ultraviolet light rays 246 from reaching port 241. Screen 240 can have multiple elements, such as porous layers 242-245, which may have different shapes and orientations to optimize ultraviolet attenuation. Moreover, each of the layers can be made to absorb ultraviolet radiation as discussed above. That is, by roughening the surface of the layers by chemically etching the inner surface of the conduit or coating the inner surface with an ultraviolet light-absorbing material.

As shown in FIG. 10, light beams emitted from an ultraviolet source are prevented from reaching port 241 because they are either reflected, absorbed, or both by screen 240.

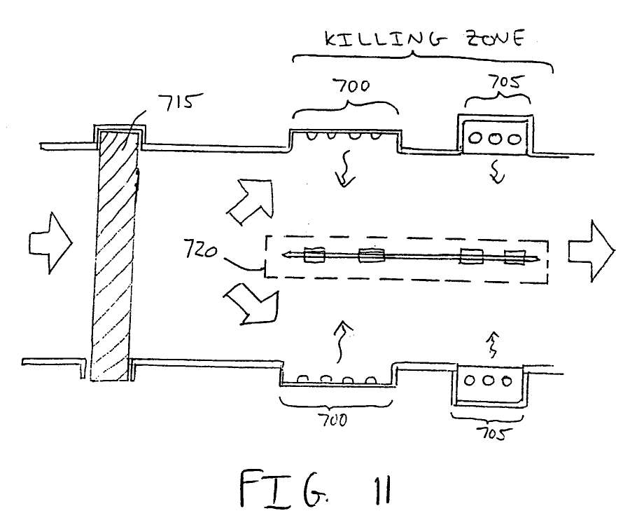

It will be appreciated that although array 500 of FIG. 5, for example, includes only solid-state light-emitting diodes, an array of light-emitting devices could also include one or more mercury vapor lamps consistent with this invention. FIG. 11, for example, shows a system in which a killing zone includes at least one solid-state light-emitting diode 700 and at least one mercury vapor lamp 705. The number of diodes and lamps should be sufficient to obtain appropriate radiation doses for the system's air flow and ambient conditions. The number of diodes and lamps should also be sufficient to cover an appropriate wavelength range for a given possible set of biohazards.

The system shown in FIG. 11 can also include filter 715, which may have multiple stages, a dust detection unit, an ozone reactive surface, etc. Preferably, at least one filter is placed before the killing zone, although filters can also be used in other locations, as desired. Also, within the killing zone, one or more photodetectors 720 can be placed to monitor the ultraviolet radiation intensity. As explained above, photodetectors 720 can be used with a unit that determines whether the diodes, lamps, and/or filters need maintenance and/or replacement. FIG. 11 does not show the electrical connections for lamps 705, diodes 700, photodetectors 720, and filter 715, but it will be appreciated that such connections are similar to the ones shown in earlier FIGS.

FIG. 15 shows another illustrative embodiment consistent with this invention for exposing biological hazards that may be present in materials, such as solid objects, to short-wavelength (ultraviolet) radiation. A system 250 includes a conveyor 255 for conveying a material 280 to be treated, wherein the conveyor has an input 260, an output 265, and a length 270. System 250 further includes at least one array 275 of solid-state light-emitting devices mounted to emit short-wavelength radiation at a material 280 while conveyed by conveyor 255 along length 270. In addition, system 250 includes at least one photodetector 285 positioned to monitor the intensity. Photodetector can be mounted, for example, on a wall near conveyor 255 or below conveyor 255 if conveyor 255 was sufficiently UV transparent (e.g., if perforated).

System 250 also can include a conveyor controller 290 for adjusting the speed of the conveyor such that material 280 is exposed to a predetermined radiation dose sufficient to neutralize the at least one biohazard. A controller 290 can base the speed of conveyor 255 on photodetector outputs. Array of devices 275 can also be controlled by an array controller 295, which can also be based on the photodetector outputs.

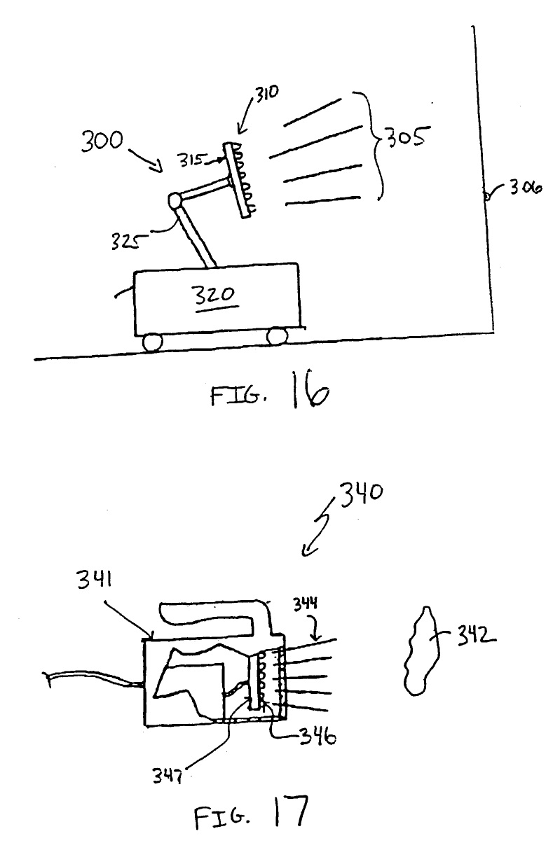

In another embodiment consistent with this invention, the array of solid-state light-emitting devices can be mobile. For example, FIG. 16 shows a mobile system 300 for exposing a material (not shown) to a directed beam 305 of ultraviolet radiation. The system can include (1) at least one mobile array 310 of solid-state light-emitting devices mounted to a structure 315 for emitting short-wavelength radiation in the form of a beam, which may be substantially collimated, or it may be converging or diverging, but having a direction; and (2) a controller, which may be located in a vehicle 320, for adjusting the direction of the beam by, for example, a controlling arm 325 on which structure 315 is mounted. The beam direction, intensity, or angle of divergence can be adjusted such that the potentially contaminated material is exposed to a predetermined radiation dose sufficient to neutralize the biohazards. For example, the beam direction can be adjusted by varying the position of controlling arm 325. Arm 325 can move in at least one spatial dimension, although it preferably moves in two or three dimensions to maximize beam exposure to contaminated surfaces.

System 300 can further include one or more remote photodetectors 306 positioned to monitor the radiation intensity at different parts of the room. In this case, the process of adjusting can be based on outputs of the photodetectors. A portion of the controller can also be remotely located from the mobile array to facilitate programming and control of the mobile array.

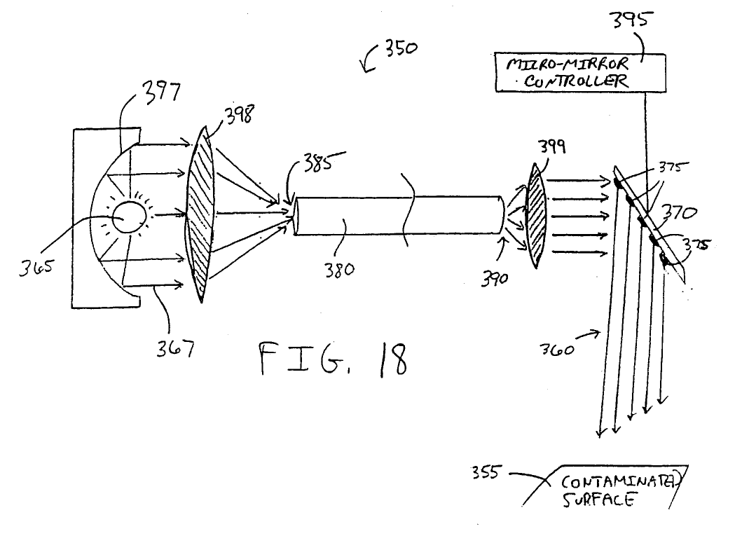

FIG. 17 shows another mobile device 340 for exposing a material 342 to a directed beam 344 of ultraviolet radiation. Device 340 can include within a housing 341 at least one mobile array 346 of solid-state light-emitting devices mounted to structure 347 for emitting short-wavelength radiation in the form of a beam, which may be substantially collimated, converging, or diverging. It will be appreciated that the beam can be shaped as necessary to achieve any desirable beam shape. Housing 341 can also include an optical system, which may be located in a vehicle 320, for adjusting the direction of the beam by, for example, varying the focus of a beam produced by array 346. The intensity or angle of divergence can also be adjusted.

FIG. 18 shows an illustrative system 350 for exposing a surface 355 to a directed beam 360 of ultraviolet radiation consistent with this invention. System 350 can include: (1) a light source 365 for emitting short-wavelength radiation 367; (2) a waveguide 380 having an input 385 and an output 390, wherein input 375 is positioned to receive at least a portion of radiation 367 and output 390 is positioned to direct that portion toward micro-mirror device 370; (3) a micro-mirror device 370 having a plurality of independently controllable mirrors 375; and (4) a micro-mirror device controller 395 coupled to micro-mirror device 370 for controlling the orientation of mirrors 375 such that surface 355 is exposed to a predetermined dose of radiation sufficient to neutralize any biohazards that may be present at surface 355. It will be appreciated, however, that a macro-mirror device, which may contain one or more mirrors, can be used instead of the micro-mirror device.

Light source 365 can be, for example, a mercury-vapor lamp, one or more light-emitting diode, or any other device, such as a laser, capable of generating a sufficient amount of UV radiation. System 350 can further include reflector 397 for reflecting radiation 367 emitted by light source 365 toward input 385 of waveguide 380 and lens 398 for further directing a portion of radiation 367 toward input 385 of waveguide 380. An optional lens 399 can be added near output 390 to direct the guided portion of radiation toward micro-mirror device 370. It will be appreciated that if light source 365 is a laser, then radiation 367 can be substantially initially collimated, making a reflector 397, lenses 398 and 399, and even waveguide 380 potentially unnecessary. Micro-mirror device 370 can include an internal or external cooling assembly, such as a plenum (not shown), which removes heat via contact with a circulating fluid, such as a liquid or a gas.

Mirror device 370 can be formed using microelectromechanical system (“MEMS”) technology. Device 370 can be controlled using, for example, DLP® control ASICS available from Texas Instruments Incorporated, of Dallas, Tex., and the like. It will be appreciated that device 370 can include one or more micro-mirror devices, programmed to coordinate radiation exposure over a surface depending, for example, on the distance between the surface and the micro-mirror device and the type of surface. For example, floors of a hospital operating room may require a higher dose than a shelf in the same operating room. It will be appreciated that individual mirrors can be programmed to move between two or more states.

In one embodiment, micro-mirror device 370 can be programmed to raster a beam over an enlarged surface area. The enlarged surface area can be an area covered by sweeping a relatively narrow beam in a series of lines (or otherwise predetermined paths) to form an area that is larger than the original beam cross-sectional area. The beam can then be programmed to return to the starting position and repeat the sweeping motion as needed.

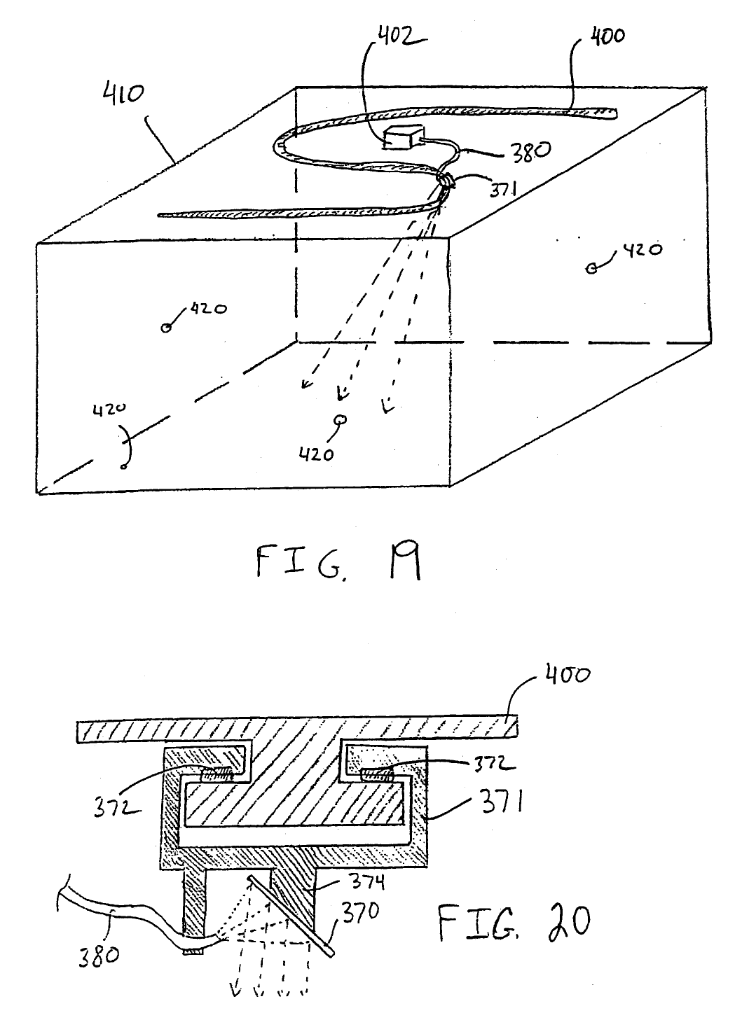

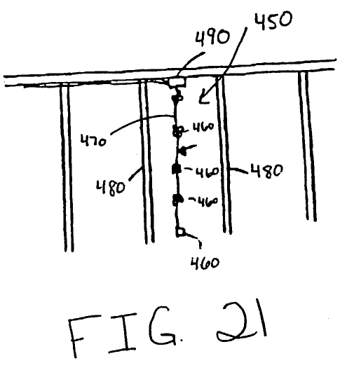

Mirror device 370 can be mobile. As shown in FIG. 19, a device 370 can be mounted on a mobile vehicle 371 that moves along a track 400. In one embodiment, a light source 365, as well as an optional reflector 397 and a lens 398, can be located in a central unit 402, which can be fixed to the ceiling, for example, of a room 410. Mirror device 370 and any accompanying hardware, such as a lens 399, can be connected via flexible waveguide 380. Mirror device controller 395 can be controlled locally or remotely through an electrical connection through track 400, a separate cord (not shown), or wireless means (not shown) to central unit 402 or any other controlling station. FIG. 20 shows a cross-section of illustrative track 400 and mobile vehicle 371, including waveguide 380 and a roller drive mechanism 372. It will be appreciated that track 400, or a separate power cord, can supply the power that powers drive mechanism 372. It will further be appreciated that supporting arm 374 that supports device 370 can be rotatable to increase the directionality of the system.

System 350 can further include one or more photodetectors 420 (FIG. 19) located at different portions of room 410. Photodetectors 420 should be sensitive to the radiation directed by device 370. Each of photodetectors 420 generates a signal indicative of the radiation intensity. The signals can be conveyed to a central unit 402, for example, and used to control the location, speed, and target surface of device 370. In this way, the mirror controller causes the mirror device to move along the track such that any desired portion of the surface is exposed to a predetermined dose of radiation. Photodetectors 420, then, generate signals that can be used in a feedback loop.

System 350 can further include a device for determining a profile of room 410 (and any objects therein) and for generating a profile information set that is used by the controller to determine a control sequence on how mobile vehicle 371 (or vehicle 320 of FIG. 16) should move along track 400 and the direction of each of the mirrors of mirror device 370. An example of a profiling device that can be used consistent with this invention is 3-D laser radar scanner unit, available from Laser Optronix AB, of Sweden.

Consistent with another aspect of this invention, a system is provided for preventing and inactivating biohazards that may reside, for example, in walls. FIG. 21 shows a system 450, for example, which includes diodes (or clusters of diodes) 460 for emitting ultraviolet radiation and a flexible carrier (such as strip 470) onto which the diodes are mounted. Strip 470 includes a power cord that supplies power to the at least one light emitting diode. System 450 can also include a controller 490 for supplying the power to the diodes periodically, continually, or a combination thereof. For example, controller 490 can supply power to LEDs such that the light is pulsed. Controller 490 can also include an appropriate voltage transformer. It will be appreciated, however, that separate controllers (e.g., located at or near each of the LEDs or clusters of LEDs) can programmed to distribute power to the LEDs individually.

Light-emitting devices, such as the devices described herein, can have a dominant wavelength below about 410 nm. The devices can be, for example, semiconductor-based light-emitting devices, such as light-emitting diodes. Wavelengths below about 410 nm are preferred because most biohazards, such as hazardous biological microorganisms, sustain damage when exposed to light having such short-wavelengths. Typically, the rate and severity of damage to living organisms increases with higher energy (i.e., shorter wavelength) radiation. This effect is even more pronounced for wavelengths below about 288 nm, which is the generally accepted cut-off caused by the ozone layer). Because living or dormant organisms have generally never been exposed to wavelengths below about 288 nm, damage usually occurs at an exponential rate upon such exposure.

Some examples of light-emitting devices that emit short-wavelength radiation consistent with this invention are listed in TABLE I.

TABLE I

| | Wavelength | Active Device | Substrate |
|---|---|---|---|
| | Range (nm) | Materials | Materials |
| Light- | 230-300 | AIN, AIN alloys | SIC, all poly- |
| emitting | | or AIN-containing | types including |
| Devices | | compounds | 4H and 6H SIC |
| | | | poly types; Al₂O₅ |
| | | | AIN, AIN alloys, |
| | | | and AIN com- |
| | | | pounds |
| | 300-400 | GaN, InGaN and | SiC, all poly-types |
| | | all other | including 4H and 6H SiC |
| | | compounds | poly-types; Al₂O₅ AIN, |
| | | containing GaN | AIN alloy, AIN- |
| Photo- | 230-390 | 6H SIC | containing compounds; |
| detectors | 280-400 | InGaN, GaN | GaN, In GaN 4H and |
| | | | 6H SiC SiC, GaN, AIN |

Light-emitting devices, such as the ones listed in TABLE I, can be used to achieve very high energy wavelength emission below 288 nm where biohazard neutralization of is particularly efficient. Certain embodiments can employ laser diodes, where directed or coherent beams are necessary. Also, the wavelength ranges shown in TABLE I, especially the shorter wavelength ranges, can be achieved using cut-off filters. Finally, it will be appreciated that when light-emitting diodes are used, they can be, for example, surface mounted or mounted in reflective cup-like structures.

The light-emitting devices can also be encapsulated, or mounted in a package, that includes lenses and other light transmission components. Suitable materials for such mechanisms include inorganic materials, organic materials, glasses, and other materials. Some of the inorganic materials that can be used consistent with this invention are BeO, B₂O₃, MgO, Al₂O₃, SiO₂, CaO, Cr₂O₃, GeO₂, SrO, Y₂O₃, ZrO₂, BaO₂, CeO₂, HfO₂, BN, AIN, Si₂N₄, MgF₂, CaF₂, SrF_{2, BaF}₂, SiC, and any combination or mixture thereof. Mixtures can be used to achieve desirable wavelength transmission ranges, transmissivities, hardnesses, and other desirable physical attributes. Some of the glasses that can be used consistent with this invention include Barium Light Flint, Crown Flint, Barium Crown, Zinc Crown, Crown, Borosilicate Crown, Dense Phosphate Crown, Phosphate Crown, and any combination or mixture thereof. Other materials that can be used to achieve particularly deep ultraviolet radiation (e.g., 210-260 nm) include Sb-doped SnO₂ (e.g., in a layer having a thickness of about 200 nm), polyanilines, and poly (cyanoterephthalylidenes).

Other embodiments of the invention will be apparent to those skilled in the art from consideration of the specification and practice of the invention disclosed herein. For example, other embodiments of the present invention may include directed beams above 300 nm to allow simple bio decontamination in household, hotel, or restaurant areas where protective clothing and glasses are used, but where failure to use such precautions would not cause a catastrophic health affects. It is intended that the specification and examples be considered as exemplary only, with a true scope and spirit of the invention being indicated by the following claims.

What is claimed is:

1. A system for exposing a fluid to ultraviolet radiation, wherein the fluid comprises at least one biohazard, the system comprising:
a conduit for conveying the fluid, wherein the conduit has an input, an output, a length, and a cross section along its length, wherein the fluid has a distribution in the conduit, and wherein the conduit is baffled so that the fluid flow is rendered more uniform while being conveyed through the conduit; and
at least one light-emitting device mounted to emit short-wavelength radiation in the conduit for neutralizing the biohazard.

2. The system of claim 1 wherein at least a portion of the conduit has an inner surface that attenuates ultraviolet light.

3. The system of claim 1 wherein the at least one device comprises at least one array of solid-state light-emitting devices.

4. The system of claim 3 wherein the conduit has at least one baffle, and at least one of the at least one array of devices is mounted on the baffle.

5. The system of claim 3 wherein the system is coupled to a fluid processing apparatus having an input and an output, wherein the system is coupled in a manner selected from a group consisting of the system input coupled to the apparatus output and the system output coupled to the apparatus input.

6. The system of claim 5 wherein the conduit is incorporated into the apparatus performing a function selected from a group consisting of air heating, air ventilating, air conditioning, air cleaning, and a combination thereof.

7. The system of claim 5 wherein the conduit is incorporated into the apparatus performing a function selected from a group consisting of liquid heating, liquid ventilating, liquid conditioning, liquid cleaning, and a combination thereof.

8. The system of claim 1 wherein the conduit has an interior surface that has at least a 50% reflectivity for the short-wavelength radiation.

9. The system of claim 1 further comprising:
a power supply supplying power at a sufficient voltage to drive the at least one array of devices; and
a controller for controlling the power to the at least one device.

10. The system of claim 1 wherein the controller comprises a cycle timer and a power switching device controlled by the timer to supply power to the at least one device.

11. The system of claim 10 wherein the cycle time supplies power to the at least one device at times selected from a group consisting of periodically, at predetermined times, and a combination thereof.

12. The system of claim 1 wherein the at least one device includes at least two arrays of devices that are substantially parallel, but not coplanar.

13. The system of claim 1 wherein the biohazard is selected from a group consisting of mold spores, microorganisms, and other biological organisms.

14. The system of claim 1 comprising a mechanism to regulate the fluid flow rate in the conduit such that the fluid is exposed to a predetermined radiation intensity for a predetermined period of time to receive a predetermined radiation dose.

15. The system of claim 1 wherein the radiation has an intensity along the length of the conduit, and wherein the system further comprises:
at least one photodetector positioned to monitor the intensity; and
a flow controller for adjusting the speed of the fluid in the conduit such that the fluid is exposed to a sufficient radiation dose to neutralize the at least one biohazard, wherein the adjusting is based on an output of the at least one photodetector.

16. The system of claim 15 further comprising:
a gas tank comprising a gas under pressure;
a pipeline in fluid communication with the gas in the tank;
a gas spout connected to the pipeline that can direct the gas toward the at least one array;
a gas valve for limiting gas flow in the pipeline; and
a gas controller coupled to the photodetector and the gas valve, wherein the photodetector generates and sends the status signal to the gas controller which opens and closes the gas valve.

17. The system of claim 1 wherein the cross section at at least one point along the length of the conduit is variable, and wherein the system further comprises a cross-sectional controller for adjusting the cross section at the at least one point such that the fluid is exposed to a sufficient radiation dose to neutralize the at least one biohazard.

18. The system of claim 1 further comprising a sorting device for physically segregating the fluid into at least a first constituent part and a second constituent part, and wherein at least one of the arrays of devices is mounted such that the first part is exposed to a different radiation intensity than the second part.

19. The system of claim 18 wherein the sorting device is selected from a group consisting of a centrifugal-force device, an electric-field device, an electro-magnetic device, a magnetic-field device, a gravitational-field device, porous screens, and any combination thereof.

20. A system for exposing a material to ultraviolet radiation, wherein the material comprises at least one biohazard, the system comprising:
a conveyor for conveying the material, wherein the conveyor has an input, an output, and a length;
at least one light-emitting device mounted to emit short-wavelength radiation at the material while being conveyed by the conveyor, wherein the radiation has an intensity along the length of the conveyor;
at least one photodetector positioned to monitor the intensity at at least one point along the length; and
a conveyor controller for adjusting the speed of the conveyor such that the material is exposed to a predetermined radiation dose sufficient to neutralize the at least one biohazard, wherein the adjusting is based on an output of the at least one photodetector.

21. The system of claim 20 wherein the at least one light-emitting device comprises an array of solid-state light emitting devices.

22. The system of claim 21 further comprising a sorting device for physically segregating the material into at least a first constituent part and a second constituent part, and wherein at least one of the arrays of devices is mounted such that the first part is exposed to a different radiation intensity than the second part.

23. The system of claim 21 wherein the sorting device is selected from a group consisting of a centrifugal-force device, an electric-field device, an electro-magnetic device, a magnetic-field device, a gravitational-field device, and any combination thereof.

24. A system for exposing a material to a directed beam of ultraviolet radiation, wherein the material comprises at least one biohazard, the system comprising:
at least one mobile light-emitting device mounted to emit short-wavelength radiation in the form of a beam having a direction; and
a controller for adjusting at least the direction of the beam such that the material is exposed to a predetermined radiation dose sufficient to neutralize the at least one biohazard.

25. The system of claim 24 wherein the at least mobile light-emitting device comprises an array of solid-state light emitting devices.

26. The system of claim 24 wherein the beam has an intensity, and wherein the system further comprises at least one remote photodetector positioned to monitor the intensity, wherein the adjusting is based on an output of the at least one photodetector.

27. The system of claim 24 wherein at least a portion of the controller is located remotely from the mobile device.

28. The system of claim 24 wherein the controller makes an adjustment selected from a group consisting of a beam direction adjustment, a beam intensity adjustment, a beam angle adjustment, and a combination thereof.

29. The system of claim 28 wherein the adjustment is made using at least one mirror.

30. A system for exposing a surface to a directed beam of ultraviolet radiation, wherein the surface has at least one biohazard, the system comprising:
a light source for emitting short-wavelength radiation in a direction;
a mirror device having at least one independently controllable mirror, wherein each mirror has a reflectivity greater than about 50% for the radiation;
a waveguide having an input and an output, wherein the input is positioned to receive at least a portion of the radiation and the output is positioned to direct toward the micro-mirror device; and
a mirror device controller coupled to the mirror device for controlling the orientation of each of the mirrors such that the surface is exposed to a predetermined radiation dose sufficient to neutralize the at least one biohazard.

31. The system of claim 30 wherein the mirror device comprises a micro-mirror device that comprises a plurality of micro-mirrors.

32. The system of claim 30 wherein the light source is selected from a group consisting of a mercury-vapor lamp, at least one light-emitting diode, and a combination thereof.

33. The system of claim 32 further comprising a reflector for reflecting radiation emitted by the light source toward the input of the waveguide.

34. The system of claim 32 further comprising a lens for directing radiation emitted by the light source toward the input of the waveguide.

35. The system of claim 32 further comprising a lens for directing radiation emitted by the light source toward the mirror device.

36. The system of claim 30 wherein the mirror device includes a cooling assembly that removes heat via contact with a fluid.

37. The system of claim 30 wherein the mirror device is mobile.

38. The system of claim 37 further comprising a track along which the mirror device can move.

39. The system of claim 38 wherein the track is mounted to a ceiling of a room and wherein the controller causes the mirror device to move along the track and causes the mirror device to direct a portion of the radiation such that any portion of the surface is exposed to a predetermined minimum dose of radiation.

40. The system of claim 39 further comprising a plurality of photodetectors located at different portions of the room, wherein at least one of the photodetectors is sensitive to the radiation and generates a signal indicative of an intensity of the radiation, wherein the controller causes the micro-mirror device to move and causes the mirror device to direct at least based on the signal.

41. The system of claim 38 wherein the track is mounted to a ceiling of a room and wherein the controller causes the mirror device to move along the track and causes the mirror device to direct a portion of the radiation such that any desired portion of the surface is exposed to a predetermined minimum dose of radiation.

42. The system of claim 38 further comprising at least one photodetector located at different portions of the room, wherein at least one of the photodetectors is sensitive to the radiation and generates a signal indicative of an intensity of the radiation, wherein the controller controls the movement and direction of the mirror device at least based on the signal.

43. The system of claim 37 further comprising a mobile vehicle for transporting the mirror device, wherein the controller causes the vehicle to move within the room and cause the micro-mirror device to direct a portion of the radiation such that any desired portion of the surface is exposed to a predetermined dose of radiation.

44. The system of claim 43 further comprising at least one photodetector located at different portions of the room, wherein the at least one photodetector is sensitive to the radiation and generates a signal indicative of an intensity of the radiation, wherein the controller controls the movement of the mobile device and direction of the mirror device at least based on the signal.

45. The system of claim 30 further comprising a device for determining a profile of the room and objects therein and for generating a profile information set that is used by the controller to determine how the mobile device and the direction of the mirror device is controlled.

46. A system for preventing and inactivating biohazards, wherein the system comprises:
at least one light emitting diode for emitting ultraviolet radiation; and
a flexible carrier onto which the at least one light emitting diode is mounted.

47. The system of claim 46 wherein the flexible carrier comprises a strip including a power cord that supplies power to the at least one light emitting diode.

48. The system of claim 47 further comprising a controller for supplying the power to the at least one diode in a manner selected from a group consisting of periodically, continually, and a combination thereof.

49. A method for exposing a material to a predetermined minimum dose of ultraviolet radiation, said method comprising:
conveying the material from an input to an output along a length;
exposing the material to short-wavelength radiation using a light-emitting device, wherein the radiation has an intensity along the length;
at least one photodetector positioned to monitor the intensity at at least one position along the length; and
adjusting the speed of the material while being conveyed such that the material is exposed to a predetermined minimum radiation dose sufficient to substantially neutralize the at least one biohazard, wherein the adjusting is based on an output of the at least one photodetector.

50. An apparatus for attenuating ultraviolet light for use with a system that inactivates biohazards using an ultraviolet light source, said system having a conduit coupled to a port, wherein the port is selected from a group consisting of an input and an output, wherein the apparatus comprises:
an ultraviolet light-absorbing surface disposed on an inner surface of the conduit.

51. The apparatus of claim 50 wherein the ultraviolet light-absorbing surface is a roughened surface.

52. The apparatus of claim 51 wherein the roughened surface is selected from a group consisting of a chemically etched surface and a coated surface.

53. The apparatus of claim 51 wherein the coated surface comprises a coating, and wherein the coating comprises:
a powder, and
a binding material.

54. The apparatus of claim 53 wherein the ultraviolet light has at least one wavelength, and the powder has a length scale on the order of the wavelength.

55. The apparatus of claim 54 wherein the powder is selected from a group consisting of a silicate glass powder, a ceramic powder, and any combination thereof.

56. A system for exposing air to ultraviolet radiation, wherein the air comprises at least one biohazard, the system comprising:
a conduit having a length and for conveying the air; and
at least one array of light-emitting devices mounted to emit short-wavelength radiation in the conduit for neutralizing the biohazard, wherein the array comprises at least two different types of ultraviolet light-emitting devices, wherein the at least two different types comprises a first type having a first peak wavelength and a second type having a second peak wavelength, wherein the first peak wavelength is different from the second peak wavelength.

57. The system of claim 56 wherein the first type of device is a mercury vapor lamp and the second type of device is a solid-state light-emitting diode.

58. The system of claim 56 wherein the first type of device is a solid-state light-emitting diode having a first peak wavelength and the second type of device is a solid-state light-emitting diode having a second peak wavelength.

59. The system of claim 56 wherein the first type of device is a mercury vapor lamp having a first optical filter with a first transmission spectrum and the second type of device is a mercury vapor lamp having a second optical filter with a second transmission spectrum.

60. The system of claim 56 further comprising a power controller for supplying power to each of the light-emitting devices according to a power distribution profile.

61. The system of claim 60 further comprising a biohazard detector coupled to the power controller, wherein the biohazard detector generates a detection signal in response to detecting a type of biohazard.

62. The system of claim 61 wherein the biohazard detector is coupled to the power controller through a communication network.

63. The system of claim 61 wherein the biohazard detector comprises a plurality of biohazard detectors, wherein each of the biohazard detectors is capable of detecting the type of biohazard.

64. The system of claim 61 wherein the power controller can, in response to receiving the detection signal, adjust the power distribution profile in accordance with the type of biohazard.

65. The system of claim 64 wherein the power controller comprises memory and wherein the power controller adjusts the distribution profile in accordance with a look-up table stored in the memory.

66. The system of claim 64 further comprising an ambient condition monitor and wherein the power controller adjusts the distribution profile in accordance with at least one monitored ambient condition.

67. The system of claim 66 wherein the ambient condition is selected from a group consisting of humidity and temperature.

68. The system of claim 56 wherein the at least two different types of light-emitting devices comprises a first type of device having a first peak wavelength between about 260 nm and about 280 nm and a second type having a second peak wavelength between about 280 nm and about 300 nm.

69. The system of claim 56 wherein the at least two different types of light-emitting devices comprises a first type of device having a first peak wavelength between about 260 nm and about 280 nm and a second type having a second peak wavelength between about 260 nm and about 280 nm.

70. The system of claim 56 having a wavelength treatment range between a lower limit and an upper limit, and wherein the at least two different types of light-emitting devices comprises a number of types of devices, each type having a different peak wavelength that is distributed between said lower and upper limits.

71. A system for exposing air to ultraviolet radiation, wherein the air comprises at least one biohazard, the system comprising:
a conduit having a killing zone, wherein the killing zone has a length in which the air is conveyed;
an array of light-emitting devices mounted to emit short-wavelength radiation in the conduit for neutralizing the biohazard;
at least one photodetector located in said conduit to sense an ultraviolet radiation intensity and generate a signal indicative of the ultraviolet radiation; and
a unit for determining, based on the at least one photodetector signal, whether any of the light-emitting devices require service.

72. The system of claim 71 further comprising a transmitter that transmits a maintenance signal to a maintenance service if any of the light-emitting devices were determined to require service.

73. The system of claim 72 wherein the maintenance signal comprises information indicative of the maintenance service required.

74. The system of claim 71 further comprising:
a filter located in series with the conduit;
a unit, coupled to the transmitter, for determining whether the filter requires replacement, wherein the transmitter transmits a replacement signal to a replacement service if any of the filter were determined to require replacement.

75. The system of claim 71 further comprising a filter located in series with the conduit, wherein the filter comprises a dust monitor that can generate a status signal indicative of an amount of dust trapped by the filter.

76. The system of claim 75 wherein the dust monitor sends the status signal to the unit when the amount of dust trapped by the filter exceeds a threshold amount.

77. The system of claim 75 wherein the dust monitor comprises:
a light-emitting diode mounted to the filter that emits a beam of light;
at least one reflective surface mounted to the filter positioned to reflect the beam of light; and
a photodetector mounted to the filter positioned to receive the beam of light after reflection from the reflective surface.

78. The system of claim 77 further comprising:
a gas tank comprising a gas under pressure;
a pipeline in fluid communication with the gas in the tank;
a gas spout connected to the pipeline that can direct the gas toward the array;
a gas valve for limiting gas flow in the pipeline; and
a gas controller coupled to the photodetector and the gas valve, wherein the photodetector generates and sends the status signal to the gas controller which opens and closes the gas valve.

79. An ozone reactive surface for use with an air processing system that inactivates airborne biohazards using an ultraviolet light source, wherein the ozone reactive surface comprises a material selected from a group consisting of an unsaturated organic polymer, a metal sulfide, a metal hydroxide, and any combination thereof.

80. The ozone reactive surface of claim 79 wherein the material comprises a metal sulfide, a metal hydroxide, and any combination thereof.
